(19) United States
(12) Patent Application Publication
(10) Pub. No.: US 2024/0066018 A1
(43) Pub. Date: Feb. 29, 2024

(54) CHRM3 AS A MARKER AND TARGET FOR CANCER THERAPY

(71) Applicant: Indivumed GmbH, Hamburg (DE)

(72) Inventors: Anton Wellstein, Washington, DC (US); Jonathan Woodsmith, Hamburg (DE)

(21) Appl. No.: 17/900,049

(22) Filed: Aug. 31, 2022

(51) Int. Cl. A61K 31/4525; A61K 31/4025; C12Q 1/6886

(52) U.S. Cl. CPC A61K 31/4525; A61K 31/4025; C12Q 1/6886; C12Q 2600/112; C12Q 2600/158

(57) ABSTRACT

The present invention relates to a method for predicting the outcome for a subject suffering from a disease. The invention further relates to a diagnostic kit for the prediction of the outcome. The invention further relates to a use of the expression of CHRM3 mRNA or protein as a marker of the outcome. The invention further relates to a method for treating a subject suffering from a disease or disorder associated with an expression of CHRM3 mRNA or protein and activity of CHRM3 protein.

### FIELD OF THE INVENTION

The present invention relates to a method for predicting the outcome for a subject suffering from a disease. The invention further relates to a diagnostic kit for the prediction of the outcome. The invention further relates to a use of the expression of CHRM3 mRNA or protein as a marker of the outcome. The invention further relates to a method for treating a subject suffering from a disease or disorder associated with an expression of CHRM3 mRNA or protein and activity of CHRM3 protein.

### BACKGROUND OF THE INVENTION

Cholinergic receptors are divided into two major classes: nicotinic acetylcholine receptors and muscarinic acetylcholine receptors based on their responsiveness to nicotine and muscarine, respectively. Unlike nicotinic acetylcholine receptors which are ion channels, muscarinic acetylcholine receptors belong to the superfamily of G-protein coupled receptors that activate ionic channels through a second messenger cascade. The muscarinic acetylcholine receptor also known as cholinergic/acetylcholine receptor M3, or the muscarinic 3, is encoded by the human gene CHRM3. Muscarinic acetylcholine receptors are divided into subtypes M1-M5 characterized by their cellular action, pharmacology, and molecular biology. They play several roles, including acting as the main end-receptor stimulated by acetylcholine released from postganglionic fibers in the parasympathetic nervous system.

Of the several subtypes of the muscarinic acetylcholine receptor, the subtype M3 muscarinic receptors are located at many places in the body, e.g in the smooth muscles of the blood vessels, as well as in the lungs. Because the M3 receptor is Gq-coupled and mediates an increase in intracellular calcium, it typically causes contraction of smooth muscle, such as that observed during bronchoconstriction and bladder voiding. However, with respect to vasculature, activation of M3 on vascular endothelial cells causes increased synthesis of nitric oxide, which diffuses to adjacent vascular smooth muscle cells and causes their relaxation, thereby explaining the paradoxical effect of parasympathomimetics on vascular tone and bronchiolar tone. The M3 receptors are also located in many glands, which help to stimulate secretion, for example, the salivary glands. Further, M3 receptors are G proteins of class Gq that upregulate phospholipase C.

Moreover, it is known that the subtype M3 muscarinic receptor activation plays an important role in promotion of colon cancer cell proliferation.

Cheng et al., Oncotarget, 2017, Vol. 8, (No. 13), 21106 describes that activation of M3 muscarinic receptor (M3R) promotes proliferation, migration and invasion of colon cancer cells. The expression of M3R in normal colon, normal colon with colon adenomas, and a colon affected by cancer (colon cancer) with primary and metastatic tumors is compared. It is concluded that M3R expression plays an important role in the early progression and invasion of colon neoplasms, but is less important when tumors have already spread.

Ali et al. Int. J. Mol. Sci. 2021, 22, 716 describes that most colorectal carcinomas over express M3 muscarinic receptors (M3R). Furthermore, activation of these receptors has been shown to stimulate cellular programs that contribute to the growth, survival, and spread of these carcinomas. In vivo studies in mouse models have demonstrated that blocking M3R expression or activation attenuates the development and progression of colon cancer. Ali et al. are investigating potential therapies targeting muscarinic receptor expression, activation, and signaling in colorectal cancer, e.g., development of muscarinic receptor antagonists, targeting of matrix metalloproteinases, M3R-stimulated EGFR transactivation, or interference with, e.g., RAS, BRAF, and components of mitogen-activated protein kinase (MAPK) signaling downstream of M3R and EGFR.

Cheng et al. Molecular Cancer 2014, 13, 77 describes the identification of a novel gene Zfp277 whose expression pattern is consistent with mediating the differential effects of CHRM3 and CHRM1 gene ablation on intestinal neoplasia in mice.

Tolaymat et al., Cancers 2019, 11, 308 describes that M3 muscarinic receptor (M3R) mRNA and protein are overexpressed in colon cancer. M3R can be activated by both traditional (e.g., acetylcholine) and non-traditional (e.g., bile acids) muscarinic ligands. This paper highlights what role key protein kinases downstream of M3R activation play in promoting the progression and spread of colon cancer.

Goto et al., Oncogene 39, 2020, 4014, describes that muscarinic receptors promote castration-resistant growth of prostate cancer. Activation of muscarinic acetylcholine receptors was studied.

Ashizawa et al., Hepatology Commun 3, 2019, 954, describes that olfactomedin 4 (OLFM4) is induced by LGR5-Wnt signaling pathway and is associated with aggressive tumor progression and poor prognosis in hepatocellular carcinoma (HCC) by regulating STAT3-induced tumor cell proliferation and cancer stem cell-like property. OLFM4 could be a prognosic predictor.

Fumagalli A., et al., Cell Stem Cell, 26, 2020, 569, describes that most colorectal cancer metastases are seeded by Lgr5″ cells.

Felton J. et al., Curr Mol. Pharmacol. 2018, 11(3), 184-190 describes the role of M3R in colon cancer.

Bernat-Peguera A. et al., Clin. Cancer Res., 27, 2021, 1491, analyzes the molecular traits underlying the response to EGFR inhibitors and the mechanism responsible for cutaneous squamous cell carcinomas. It is suggested that a combined EGFR- and FGFR-targeted therapy may be used to treat cutaneous squamous cell carcinomas.

Calaf G. M. et al., Cancer 14, 2022, 2322, 1 reports about the correlation between the growth factors and M3 receptors, the survival differences adjusted by staging of clinical factor and the association between gene expression and immune infiltration levels in different human cancers.

Houghton L. A. et al., Aliment Pharm. Therap., 1997, 11, 561 discloses that Zamifenacin can act as an M3 selective muscarinic antagonist in order to reduce colonic motor activity in patients with irritable bowel syndrome.

Steers W. D. et al., Urol. Clin. N. Am., 2006, 33, 475, describes darifenacin as an antimuscarinic for the treatment of overactive bladder and urge urinary incontinence.

US2017/0246267 relates to a method for treating gastric cancer or colon cancer in a subject by administering a cholinergic antagonist, a Botulinum toxin, a NGF inhibitor, a TRK inhibitor, or performing a surgical denervation. This document does not give any hint for a person skilled in the art that the gene expression of M3 and additional genes (correlated genes) forms a prognostic signature that allows to predict the outcome for a patient. Further, this document does not give any hint that it is also possible to form a treatment signature based on the finding that sensitivity to treatment with a M3 inhibitor or a combination of M3 inhibitor and other inhibitors depends on the expression and/or co-expression of M3 response (or activity) genes.

It is an object of the present invention to provide a method for predicting the outcome for a subject suffering from a disease or disorder based on CHRM3 expression.

In particular, it is an object of the present invention to provide a method for predicting if the outcome of a patient suffering from a cancer is likely poor or good. Especially, the method is applied to patients having a cancer of epithelial cell origin, in particular colorectal carcinoma.

Furthermore, it is an object of the present invention to provide a method for treating a subject suffering from a disease or disorder based on CHRM3 mRNA over expression and CHRM3 protein activity. In particular, it is an object to select patients with the M3 response signature for treatment and exclude patients who do not have that signature.

The method according to the invention has the following advantages:
The method helps to decide for patients on further treatment measures and/or surveilance as an alternative to nonintervention.
The method allows to select various collectives of patients, e.g. patients that do not need further treatment and/or surveilance, patients that may benefit from further treatment and/or surveilance and patients that in view of the decision made in accordance with the guidelines should be subjected to further curative or palliative treatment or can be spared such treatment.
In tumors with elevated levels of CHRM3 it is possible to determine the activity of CHRM3 to decide whether or not to treat the subject with an inhibitor of CHRM3. The activity of CHRM3 can be determined from altered levels of expression of genes that respond to CHRM3 stimuli.

### SUMMARY OF THE INVENTION

The invention relates to a method for predicting the outcome for a subject suffering from a disease or disorder based on the expression level of CHRM3 comprising the steps:
a) collecting a sample from a tumor of a subject having a cancer of epithelial cell origin;
b) determining the level of expression of CHRM3 mRNA or CHRM3 protein;
c) comparing the level of expression of CHRM3 mRNA or CHRM3 protein to a control level;

wherein a significantly elevated level of expression of CHRM3 mRNA or CHRM3 protein compared to the control level indicates that the subject is likely to experience a poor outcome of the disease.

The invention further relates to a kit for predicting the outcome for a subject suffering from a disease or disorder based on the expression level of CHRM3 mRNA or CHRM3 protein and optionally of at least one further gene correlated with CHRM3 mRNA expression, preferably selected from LGR5, OLFM4, TRAF5, CD46, TGFB1, SLC5A1 and OXGR1 comprising means for determining the level of expression of CHRM3 mRNA or CHRM3 protein and optionally at least one gene correlated therewith.

The invention further relates to a use of the expression of CHRM3 mRNA or CHRM3 protein as a marker of the outcome for a subject suffering from a disease or disorder and optionally by using at least one further gene selected from LGR5, OLFM4, TRAF5, CD46, TGFB1, SLC5A1 and OXGR1.

The invention further relates to a method for treating a subject suffering from a disease or disorder associated with an over expression of CHRM3 mRNA or CHRM3 protein comprising the steps:
a) collecting a sample from a tumor of a subject having a cancer of epithelial cell origin;
b) determining the level of expression of CHRM3 mRNA or CHRM3 protein;
c) comparing the level of expression of CHRM3 mRNA or CHRM3 protein to a control level;

if the level of the expression of CHRM3 mRNA or CHRM3 protein is significantly elevated compared to the control level the subject is subjected to an anti tumor treatment.

The invention further relates to a method for treating a subject suffering from a disease or disorder associated with an over expression of CHRM3 mRNA or CHRM3 protein and activity of CHRM3 protein, comprising the steps:
a) collecting a sample from a tumor of a subject having a cancer of epithelial cell origin;
b) determining the level of expression of CHRM3 mRNA or CHRM3 protein and activity of CHRM3 protein;
c) comparing the level of expression of CHRM3 mRNA or CHRM3 protein and activity of CHRM3 protein to a control level;

if the level of the expression of CHRM3 mRNA or CHRM3 protein and activity of CHRM3 protein is significantly elevated compared to a control level the subject is subjected to treatment with an effective amount of an agent that inhibits CHRM3 activity.

### BRIEF DESCRIPTION OF THE DRAWINGS

FIG. 1 shows an increased expression of CHRM3 in tumor samples in comparison to normal samples as measured by RNA-Seq. The tumor samples (boxplot in dark grey/right) show a statistically significant upregulation in comparison to the normal samples;

FIG. 2 shows primary colon cancer samples from patients with high M3 receptor expression were grown as organoids in cell culture in the absence of the typically added exogenous growth factors EGF and FGF. Organoid growth was inhibited by M3-selective antagonist zamifenacin in a concentration range commensurate with the M3 receptor affinity of the drug. The global M-receptor agonist carbachol added on top of the antagonist was able to reverse the inhibition;

FIG. 3A is a Kaplan meier curve based on progression free survival and the expression of CHMR3 in tumor patients wherein the patients were split into two cohorts based on the median expression of CHRM3 in the tumor patients. The bottom line represents the group of patients that have an above median expression of CHMR3, and the top line represents patients who have a below median expression of CHRM3. As can be seen in the figure, the patient group with higher expression are associated with shorter survival times; and

FIG. 3B is a Kaplan meier curve based on progression free survival and the expression of CHMR3 in tumor patients wherein the patients were split into two cohorts based on the upper and lower quartile expression of CHRM3 in the tumor patients. The bottom line represents the group of patients that have top 25% CHMR3 expression, and the top line represents patients how have the lowest 25% CHRM3 expression. As can be seen in the figure, the patient group with higher expression are associated with shorter survival times.

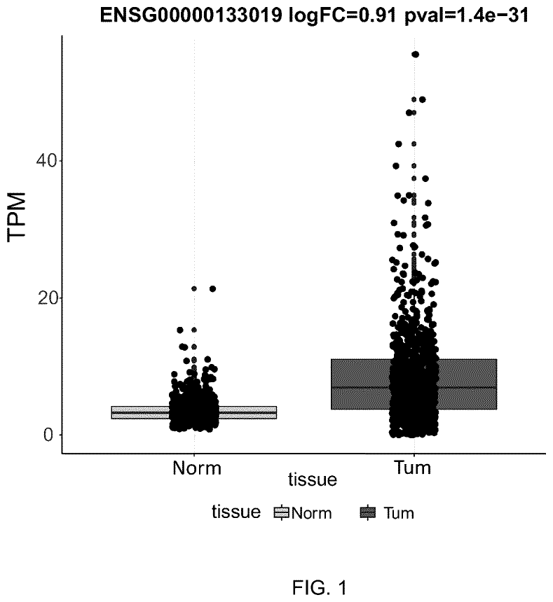

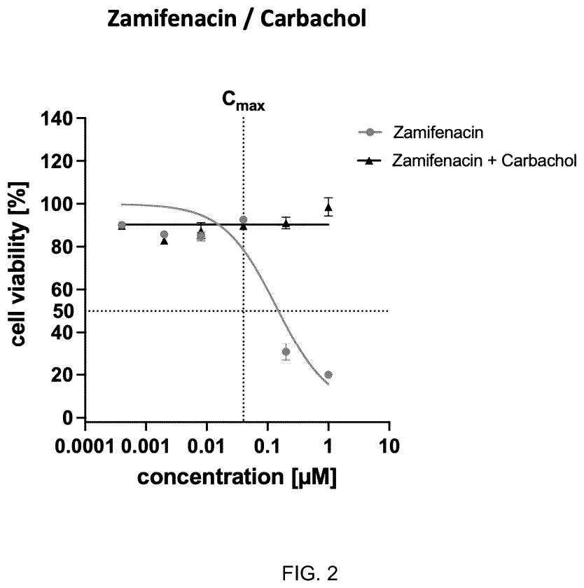

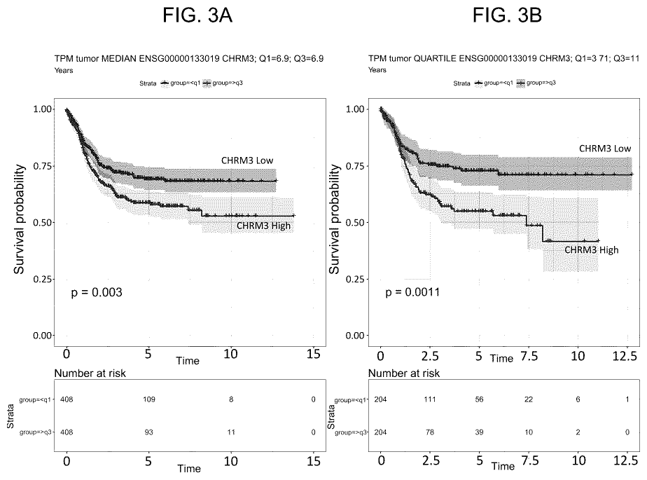

### DETAILED DESCRIPTION OF THE INVENTION

“Protein” is used interchangeably with polypeptide, and includes protein fragments and domains as well as whole proteins.

In the sense of the invention the term “subject” refers to any human or animal. A (non-human) animal includes all vertebrates, e.g. mammals and non-mammals, including cows, sheep, pigs, goats, horses, poultry, dogs, cats, non-human primates, rodents etc. In one embodiment, the subject is a human subject.

The term “marker” as used herein refers to a gene, the expression level of which, is measured using a gene product.

The gene expression of M3 (CHRM3 mRNA or protein expression) and of additional genes (so called “correlated genes”) forms a prognostic signature (fingerprint) that allows to predict the outcome for a patient. This prognostic signature can be used to set up a prognostic rating scale.

The expression of M3 (CHRM3 mRNA or protein expression) and additional genes (so called “response genes”) forms a treatment signature that is characteristic for the sensitivity of a subject to tumor treatment, in particular to treatment with an M3 inhibitor or a combination of M3 inhibitor and other inhibitors. This therapeutic signature can be used to set up a therapeutic rating scale.

In other words, correlated genes are selected from a group of genes whose expression correlates with CHRM3 mRNA or CHRM3 protein expression. Response genes are selected from a group of genes that are controlled by CHRM3 protein activity. Correlated genes and response genes are selected from a group of genes but are not necessarily identical.

Correlated genes can be derived from measuring genes expressed in cancer biopsies or samples from patients. In patients with high or low CHRM3 expression the expression level of CHRM3 correlated genes will be used to add weight to the expression level of CHRM3.

Response genes are derived from experimentally inhibiting CHRM3 in human cancer cells that express CHRM3 and measuring changes in gene expression due to the inhibition of CHRM3. For this, cancer cells are kept in culture and CHRM3 activity is inhibited. Inhibition is achieved by small molecule inhibitors of CHRM3 (examples are Darifenacin and Zamifenacin) or by knockdown of CHRM3 mRNA that will deplete the CHRM3 protein from cells. Cancer cells are collected before and at different times after inhibition of CHRM3. Typical time points are after 1, 2, 3, 4, 5, 6 or 7 days. In the collected cancer cells gene expression is measured by RNA sequencing that generates a readout of the whole transcriptome. Expression of the whole transcriptome after inhibition of CHRM3 is compared to control levels and sets of genes are identified by various established methods. Changes in gene expression after CHRM3 inhibition reveals which genes are indicative of CHRM3 activity. These genes constitute the response gene signature.

Expression of CHRM3 is defined by the level of CHRM3 mRNA or protein detected in a sample. The level of CHRM3 mRNA can be measured by quantitative RT-PCR or in situ hybridization or RNA sequencing. The determination of the level of expression of the CHRM3 protein can be done by using antibodies, immunohistochemistry, ELISA assays or mass specrometry.

Activity of CHRM3 is defined as an active state caused by ligand binding and/or over expression leading to expression of CHRM3 response genes and proteins and can be detected by quantitative RT-PCR or RNA sequencing.

In the sense of the invention the term “outcome” is the result of a treatment or series of treatments of a certain disease.

The outcome is determined at a point in time during or after the treatment on the basis of one criterion or a set of several criteria. Consideration of outcome helps to determine the effectiveness and appropriateness of medical interventions and to evaluate them in relation to alternatives, particularly nonintervention.

There are a number of defintions of the outcome of a disease, defined e.g. by using different endpoints. One way of determining the outcome is the “long-term” survival that refers to survival after diagnosis and/or initial treatment for a particular time period, e.g., for at least 3 years. Another way is the “Recurrence-Free Survival” (RFS) that refers to survival for a time period (usually in years) from diagnosis and/or initial treatment to cancer recurrence or death due to recurrence of cancer. Another way of determining is the “Overall Survival” (OS) that refers to the time (in years) from diagnosis and/or initial treatment to death from any cause. A further way is “Disease-Free Survival” (DFS) that refers to survival for a time period (usually in years) from diagnosis and/or initial treatment to first cancer recurrence or death from any cause.

“Outcome analyses” often focus on changes in quality of life; the respective preventive or therapeutic measures are thus to be evaluated in a more meaningful way for future subjects than is possible with the so-called surrogate markers, parameters or endpoints (these are measurable variables that are not directly relevant for the person concerned, such as measurement and laboratory values, tumor diameter).

Instead of surrogate parameters or an intuitive description of the case (“cured”/“not cured” or similar), a description of the overall situation should be as precisely defined as possible.

Fibroblast growth factor (FGF) is a protein that stimulates cell growth and differentiation. The FGF-receptor (FGFR) binds to FGF and forms a protein-ligand interaction.

Epidermal growth factor (EGF) is a protein that stimulates cell growth and differentiation. The EGF-receptor (EGFR) binds to EGF and forms a protein-ligand interaction.

Leucine-rich repeat-containing G-protein coupled receptor 5 (LGR5) also known as G-protein coupled receptor 49 (GPR49) or G-protein coupled receptor 67 (GPR67) is a protein that in humans is encoded by the LGR5 gene.

Olfactomedin 4 is a protein that in humans is encoded by the OLFM4 gene.

TNF receptor-associated factor 5 is a protein that in humans is encoded by the TRAF5 gene.

CD46 complement regulatory protein also known as CD46 (cluster of differentiation 46) and Membrane Cofactor Protein is a protein which in humans is encoded by the CD46 gene.

Transforming growth factor beta 1 or TGF-01 is a polypeptide member of the transforming growth factor beta superfamily of cytokines. In humans, TGF-01 is encoded by the TGFB1 gene.

Sodium/glucose cotransporter 1 (SGLT1) is a protein in humans that is encoded by the SLC5A1 gene which encodes the production of the SGLT1 protein to line the absorptive cells in the small intestine and the epithelial cells of the kidney tubules of the nephron for the purpose of glucose uptake into cells.

2-Oxoglutarate receptor 1 (OXGR1), also known as cysteinyl leukotriene receptor E (CysLTE) and GPR99, is a protein that in humans is encoded by the OXGR1 gene.

A first embodiment relates to a method for predicting the outcome for a subject suffering from a disease or disorder based on the expression level of CHRM3 mRNA.

### Step a)

In step a) a sample from a tumor in a subject having a cancer of epithelial cell origin is collected.

Suitable samples contain tissues or cells from the tumor of a subject having a cancer of epithelial cell origin. The sample preferably comprises a biopsy sample, such as a tumor biopsy, a primary tissue, a metastatic tissue. In particular, the specimen can be obtained by needle biopsy, image-guided biopsy, surgical (excisional) biopsy, shave/punch biopsy, endoscopic biopsy, laparoscopic biopsy and combinations thereof.

### Step b) and Step c)

In step b) of the method according to the invention the level of expression of CHRM3 mRNA or protein is determined.

The level of expression of protein can be determined inter alia by targeted mass specrometric analysis on tumor tissue.

The determination of the level of expression of the CHRM3 protein can be done by using antibodies or other appropriate techniques such as immunohistochemistry, ELISA assays, mass specrometry. The level of CHRM3 mRNA can be measured by quantitative RT-PCR or in situ hybridization or RNA sequencing.

The antibodies for use in the invention may be produced by any suitable method known in the art. Such antibodies include, but are not limited to, polyclonal, monoclonal, humanized, phage display-derived antibodies or chimeric antibodies.

In step c) of the method according to the invention the level of expression of CHRM3 mRNA or CHRM3 protein is compared to a control level.

Thereby, a significantly elevated level of expression of CHRM3 mRNA or CHRM3 protein compared to the control level indicates that the subject is likely to experience a poor outcome of the disease. In particular, the level of expression of CHRM3 mRNA or CHRM3 protein can be used in a rating scale (prognostic rating scale) as defined above and below that is used to decide, whether a subject is likely to experience a poor outcome and should be subjected to a treatment and/or surveilance.

The control level refers to the level of expression of a protein in a non-cancerous tissue. In other words the control level refers to the level of expression of a protein in a normal, non-tumorous tissue. It refers to the normalized level of a gene product, e.g. the normalized value determined for the RNA expression level of a gene or for the polypeptide expression level of a gene.

A “significantly elevated” level of expression (compared to a control level) is a level whose difference from the control level is statistically significant, using statistical methods that are appropriate and well-known in the art.

Methods for measuring the level of expression are conventional and routine. In general, the measurement relies on the existence of sets of antibodies that are e.g. specific for over expression of protein. Such antibodies are commercially available or can be generated routinely, using conventional procedures.

Assays to measure the activity and/or level of expression can be readily adapted to high throughput formats, e.g. using robotics, if desired.

Preferably, step b) and step c) additionally comprises determining the level of expression of at least one gene correlated with CHRM3 mRNA expression and comparing the level of expression of the at least one gene correlated with CHRM3 mRNA expression with a control level.

In particular, genes correlated with CHRM3 mRNA expression are selected from LGR5, OLFM4, TRAF5, CD46, TGFB1, SLC5A1 and OXGR1.

Thereby, a significantly elevated level of expression of at least one gene correlated with CHRM3 mRNA or CHRM3 protein expression compared to the control level is a further indicator that the subject is likely to experience a poor outcome of the disease. In particular, the level of expression of at least one correlated gene can be used additionally in a rating scale (prognostic rating scale) as defined below that is used to decide, whether a subject is likely to experience a poor outcome and should be subjected to a treatment and/or surveilance.

Preferably, the level of expression of CHRM3 mRNA and at least one further correlated gene is converted into a rating scale (prognostic rating scale).

Advantageously, in the method of the present invention it is possible to establish a rating scale based on the level of expression of CHRM3 mRNA and/or CHRM3 protein and optionally at least one further correlated gene. Advantageously, this rating scale can be used to decide, whether the subject is likely to experience a poor outcome. In particular, the level of expression of CHRM3 mRNA and/or CHRM3 protein and optionally at least one further correlated gene can be used to decide, whether a subject should be subjected to a treatment by anti tumor therapy and/or surveilance of progress of the disease. This method allows a decision at a very early stage of the disease, where markers used in state of the art staging protocols (like e.g. the occurrence of other tumor markers or metastases), usually are not yet detectible.

In a second embodiment, the invention relates to a kit for predicting the outcome for a subject suffering from a disease or disorder based on the expression level of CHRM3 mRNA or CHRM3 protein and optionally of at least one further gene correlated with CHRM3 mRNA or CHRM3 protein expression comprising means for determining the level of expression of CHRM3 mRNA or CHRM3 protein and optionally at least one gene correlated therewith.

Preferably, at least one further gene correlated with CHRM3 mRNA expression is selected from LGR5, OLFM4, TRAF5, CD46, TGFB1, SLC5A1 and OXGR1.

A third embodiment of the invention relates to the use of the expression of CHRM3 mRNA or CHRM3 protein as a marker of the outcome for a subject suffering from a disease or disorder based on the expression level of CHRM3 mRNA or CHRM3 protein by determining the expression level of CHRM3 mRNA or CHRM3 protein and at least one gene correlated with CHRM3 mRNA expression.

Preferably, at least one further gene correlated with CHRM3 mRNA expression is selected from LGR5, OLFM4, TRAF5, CD46, TGFB1, SLC5A1 and OXGR1.

A fourth embodiment of the invention relates to a method for treating a subject suffering from a disease or disorder associated with an over expression of CHRM3 mRNA or CHRM3 protein. If the level of the expression of CHRM3 mRNA or CHRM3 protein is significantly elevated compared to the control level the subject is subjected to an anti tumor treatment.

There is a great variety of anti tumor therapies that are well known to a person skilled in the art. Based on specific factors like nature of the cancer, the stage of disease etc. the skilled person can decide which therapy is appropriate for treatment of a subject in need of an anti tumor therapy e.g. surgery, radiotherapy and/or chemotherapy.

Preferably, step b) and step c) additionally comprises determining the level of expression of at least one gene correlated to CHRM3 mRNA or CHRM3 protein expression, preferably selected from LGR5, OLFM4, TRAF5, CD46, TGFB1, SLC5A1 and OXGR1 and comparing the level of expression of that gene with a control level.

For the decision whether a certain subject is subjected to treatment by anti tumor therapy and/or surveilance the aforementioned rating scale (prognostic rating scale) can be employed.

Preferably, the level of expression of CHRM3 mRNA and at least one further correlated gene is converted into a rating scale.

A fifth embodiment of the invention relates to a method for treating a subject suffering from a disease or disorder associated with over expression of CHRM3 mRNA or CHRM3 protein and activity of the CHRM3 protein. If the level of the expression of CHRM3 mRNA or CHRM3 protein and activity of CHRM3 protein is significantly elevated compared to the control level the subject is subjected to treatment with an effective amount of an agent that inhibits CHRM3 activity. Cancer-relevant activity of CHRM3 will be determined by measuring expression of genes that indicate activity of CHRM3. These genes are determined by inhibiting CHRM3 in patient-derived cancer cells and measuring which genes are altered significantly after the inhibition. This is a CHRM3 therapeutic signature.

Preferably, step b) and step c) additionally comprises determining the level of expression of at least one response gene to the CHRM3 protein activity, and comparing the level of expression of that gene with a control level.

For the decision whether a certain subject is subjected to treatment by an agent that inhibits CHRM3 activity a rating scale (therapeutic rating scale) can be employed.

Preferably, the level of expression of CHRM3 mRNA and activity of the CHRM3 protein and at least one further response gene is converted into a rating scale (therapeutic rating scale).

Preferably, the subject is administered with an additionally effective amount of an EGFR inhibitor and/or FGFR inhibitor.

Suitable EGFR inhibitors are preferably selected from erlotinib, gefitinib, osimertinib, cetuximab and panitumumab.

Suitable FGFR inhibitors are preferably selected from erdafitinib, pemigatinib and infigratinib.

One skilled in the art will appreciate that the particular formulation will depend, in part, upon the particular inhibitory agent or other chemotherapeutic agent, that is employed, and the chosen route of administration. Accordingly, there is a wide variety of suitable formulations of compositions. Formulations suitable for oral, parenteral, aerosol, transdermal, topical, or other forms of administration will be evident to the skilled person.

One skilled in the art can easily determine the appropriate dose, schedule, and method of administration for the exact formulation of the composition being used, in order to achieve the desired anti-cancer effective amount or effective concentration of the agent in the individual patient. One skilled in the art also can readily determine and use an appropriate indicator of the “effective concentration”/“effective amount” of the inhibitors by a direct or indirect analysis of appropriate patient samples (e.g., blood and/or tissues).

The dose of an inhibitory agent, or composition thereof, administered to an animal, particularly a human, should be sufficient to effect at least a therapeutic response in the individual over a reasonable time frame (an anticancer effective amount). The exact amount of the dose will vary from subject to subject, depending on the species, age, weight and general condition of the subject, the severity or mechanism of any disorder being treated, the particular agent or vehicle used, its mode of administration and the like. The dose used to achieve a desired anticancer concentration in vivo will be determined by the potency of the particular inhibitory agent employed, the pharmacodynamics associated with the agent in the host, the severity of the disease state of infected individuals, as well as, in the case of systemic administration, the body weight and age of the individual. The size of the dose also will be determined by the existence of any adverse side effects that may accompany the particular inhibitory agent, or composition thereof, employed. It is generally desirable, whenever possible, to keep adverse side effects to a minimum.

Preferably, the disease or disorder from which the subject is suffering is cancer, in particular cancer of epithelial cell origin. This cancer includes primary cancer and secondary (metastatic) disease.

In particular, the cancer is selected from glioblastoma, melanoma, colon, colorectal, lung, breast, ovary, prostrate, stomach, pancreas, bladder, head, neck, and kidney cancer.

Especially, the disease or disorder is colorectal carcinoma.

Preferably, the CHRM3 mRNA inhibitor is selected from small molecule inhibitors, monoclonal antibodies and a combination thereof. Small molecule inhibitors are preferably selected from darifenacin and zamifenacin.

### REFERENCE LISTING

Cheng et al., Oncotarget, 2017, Vol. 8, (No. 13), 11106
Ali et al., Int. J. Mol. Sci., 2021, 22, 716
Cheng et al. Molecular Cancer, 2014, 13, 77
Tolaymat et al., Cancers, 2019, 11, 308
Goto et al., Oncogene, 39, 2020, 4014
Ashizawa et al., Hepatology Commun, 3, 954-970, 2019, 954
Fumagalli A., et al., Cell Stem Cell, 26, 2020, 569
Felton J. et al., Curr Mol. Pharmacol., 2018, 11(3), 184-190
Bernat-Peguera A. et al., Clin. Cancer Res., 27, 2021, 1491,
Calaf G. M. et al., Cancer, 14, 2022, 2322, 1
Houghton L. A. et al., Aliment Pharm. Therap., 1997, 11, 561
Steers W. D. et al., Urol. Clin. N. Am., 2006, 33, 475
US 2017/0246267

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method for predicting the outcome for a subject suffering from a disease or disorder based on the expression level of CHRM3 mRNA, comprising the steps:
a) collecting a sample from a tumor of a subject having a cancer of epithelial cell origin;
b) determining the level of expression of CHRM3 mRNA or CHRM3 protein;
c) comparing the level of expression of CHRM3 mRNA or CHRM3 protein to a control level;
wherein a significantly elevated level of expression of CHRM3 mRNA or protein compared to the control level indicates that the subject is likely to experience a poor outcome of the disease.

2. The method of claim 1, wherein step b) and step c) additionally comprises determining the level of expression of at least one gene correlated with CHRM3 mRNA expression, preferably selected from LGR5, OLFM4, TRAF5, CD46, TGFB1, SLC5A1 and OXGR1 and comparing the level of expression of at least one gene correlated with CHRM3 mRNA expression with a control level.

3. The method of claim 1, wherein the level of expression of CHRM3 mRNA and optionally at least one further correlated gene is converted into a (prognostic) rating scale.

4. The method of claim 3, wherein the (prognostic) rating scale is used to decide whether the subject is subjected to a treatment by anti tumor therapy and/or surveillance of progress of the disease.

5. (canceled)

6. (canceled)

7. A method for treating a subject suffering from a disease or disorder associated with an over expression of CHRM3 mRNA or CHRM3 protein, comprising the steps:
a) collecting a sample from a tumor of a subject having a cancer of epithelial cell origin;
b) determining the level of expression of CHRM3 mRNA or CHRM3 protein;
c) comparing the level of expression of CHRM3 mRNA or CHRM3 protein to a control level;
if the level of the expression of CHRM3 mRNA or CHRM3 protein is significantly elevated compared to the control level the subject is subjected to an anti tumor treatment.

8. The method according to claim 7, wherein step b) and step c) additionally comprises determining the level of expression of at least one gene correlated with CHRM3 mRNA or CHRM3 protein expression, preferably selected from LGR5, OLFM4, TRAF5, CD46, TGFB1, SLC5A1 and OXGR1, and comparing the level of expression of the at least one gene correlated with CHRM3 mRNA expression with a control level.

9. The method of claim 7, wherein the level of expression of CHRM3 mRNA or CHRM3 protein and optionally at least one further correlated gene is converted into a (prognostic) rating scale and the (prognostic) rating scale is used to decide, whether the subject is subjected to a treatment by anti tumor therapy and/or surveillance of progress of the disease.

10. A method for treating a subject suffering from a disease or disorder associated with an over expression of CHRM3 mRNA or CHRM3 protein and activity of CHRM3 protein, comprising the steps:
a) collecting a sample from a tumor of a subject having a cancer of epithelial cell origin;
b) determining the level of expression of CHRM3 mRNA or CHRM3 protein and activity of CHRM3 protein;
c) comparing the level of expression of CHRM3 mRNA or CHRM3 protein and activity of CHRM3 protein to a control level;
if the level of the expression of CHRM3 mRNA or CHRM3 protein and activity of CHRM3 protein is significantly elevated compared to a control level the subject is subjected to treatment with an effective amount of an agent that inhibits CHRM3 activity.

11. The method according to claim 10, wherein step b) and step c) additionally comprises determining the level of expression of at least one response gene to the CHRM3 protein activity, and comparing the level of expression of the at least one response gene with a control level.

12. The method according to claim 10, wherein the level of CHRM3 protein activity and optionally at least one further response gene is converted into a (therapeutic) rating scale and the (therapeutic) rating scale is used to decide, whether the subject is subjected to a treatment with an effective amount of an agent that inhibits CHRM3 activity.

13. The method according to claim 10, wherein additionally an effective amount of an EGFR inhibitor and/or FGFR inhibitor is administered.

14. The method according claim 10, wherein the cancer includes primary and secondary metastatic disease.

15. The method according to claim 10, wherein the cancer is selected from colorectal carcinoma, glioblastoma, a melanoma, cancer of the lung, breast, ovary, stomach, pancreas, bladder, head, neck, colon and kidney.

16. The method according to claim 10, wherein the subject is a human.

17. The method according to claim 10, wherein the CHRM3 mRNA inhibitor is selected from small molecule inhibitors and monoclonal antibodies or a combination thereof.

18. The method according to claim 10, wherein the subject is a human patient suffering from colorectal carcinoma, and the CHRM3 mRNA inhibitor is selected from darifenacin or zamifenacin and mixtures thereof.
